(19) United States
(12) Patent Application Publication
(10) Pub. No.: US 2012/0129236 A1
(43) Pub. Date: May 24, 2012

(54) FORMULATION AND PROCESS FOR CO2 CAPTURE USING AMINO ACIDS AND BIOCATALYSTS

(75) Inventors: Sylvie Fradette, Pintendre (CA); Julie Gingras, Quebec (CA); Jonathan Carley, Calgary (CA); Glenn R. Kelly, Trois-Rivieres (CA); Olivera Ceperkovic, Quebec (CA)

(73) Assignee: CO2 SOLUTIONS INC., Quebec, QC (CA)

(21) Appl. No.: 13/388,854

(22) PCT Filed: Aug. 4, 2010
(86) PCT No.: PCT/CA2010/001212

§ 371 Date: Feb. 3, 2012

Related U.S. Application Data
(60) Provisional application No. 61/231,039, filed on Aug. 4, 2009.

(51) Int. Cl. C09K 3/00; C12P 3/00

(52) U.S. Cl. 435/168; 252/190

(57) ABSTRACT

A formulation and a process for CO₂ capture, where a CO₂-containing gas in contacted with water, biocatalyst and an amino acid compound, enabling the dissolution and transformation of the CO₂ into bicarbonate ions and hydrogen ions, producing an ion-rich solution and a CO₂-depleted gas. The amino acids may present slow absorption kinetics and having elevated stability such that absorption is enhanced in combination with the biocatalyst. The amino acid compound and the biocatalyst may be selected such that the active sites of the biocatalyst benefit from proton removal facilitated by the amino acid compounds, thus improving the CO₂ absorption.

### FIELD OF INVENTION

The present invention relates generally to CO₂ capture and more particularly to a formulation and a process for CO₂ capture using amino acids and biocatalysts.

### BACKGROUND OF THE INVENTION

Increasingly dire warnings of the dangers of climate change by the world's scientific community combined with greater public awareness and concern over the issue has prompted increased momentum towards global regulation aimed at reducing man-made greenhouse gas (GHGs) emissions, most notably carbon dioxide. Ultimately, a significant cut in North American and global CO₂ emissions will require reductions from the electricity production sector, the single largest source of CO₂ worldwide. According to the International Energy Agency's (IEA) GHG Program, as of 2006 there were nearly 5,000 fossil fuel power plants worldwide generating nearly 11 billion tons of CO₂, representing nearly 40% of total global anthropogenic CO₂ emissions. Of these emissions from the power generation sector, 61% were from coal fired plants. Although the long-term agenda advocated by governments is replacement of fossil fuel generation by renewables, growing energy demand, combined with the enormous dependence on fossil generation in the near to medium term dictates that this fossil base remain operational. Thus, to implement an effective GHG reduction system will require that the CO₂ emissions generated by this sector be mitigated, with carbon capture and storage (CCS) providing one of the best known solutions.

The CCS process removes CO₂ from a CO₂ containing flue gas, enables production of a highly concentrated CO₂ gas stream which is compressed and transported to a sequestration site. This site may be a depleted oil field or a saline aquifer. Sequestration in ocean and mineral carbonation are two alternate ways to sequester that are in the research phase. Captured CO₂ can also be used for enhanced oil recovery.

Current technologies for CO₂ capture are based primarily on the use of amines solutions which are circulated through two main distinct units: an absorption tower coupled to a desorption (or stripping) tower.

A very significant barrier to adoption of carbon capture technology on large scale is cost of capture. Conventional CO₂ capture with available technology, based primarily on the use of amine solvents, is an energy intensive process that involves heating the solvent to high temperature to strip the CO₂ (and regenerate the solvent) for underground sequestration. The conventional use of amines involves an associated capture cost of approximately US $60 per ton of CO₂ (IPCC), which represents approximately 80% of the total cost of carbon capture and sequestration (CCS), the remaining 20% being attributable to CO₂ compression, pipelining, storage and monitoring. This large cost for the capture portion has, to present, made large scale CCS unviable; based on data from the IPCC, for instance, for a 700 megawatt (MW) pulverized coal power plant that produces 4 million metric tons of CO₂ per year, the capital cost of conventional CO₂ capture equipment on a retrofit basis would be nearly $800 million and the annual operating cost and plant energy penalty would be nearly $240 million. As such, there is a need to reduce the costs of the process and develop new and innovative approaches to the problem.

Amino acids are molecules containing at least one amino group and one carboxylic group. Accordingly, and as is the case with amines, amino acids can be separated into three classes; primary, secondary and tertiary. Their CO₂ capture and desorption performance is also generally comparable to amines; primary amino acids are kinetically rapid for capture and have higher energies of desorption whereas tertiary amino acids are slower on capture but present more favourable energetics for desorption. The main advantages of amino acids over amines are that they are generally more stable, they are biodegradable and have no vapour pressure. However, the kinetically rapid amino acids are unstable for industrial CO₂ capture operations whereas stable amino acids are quite slow for capture.

Amino acids react with CO₂ in a similar fashion to amines, i.e. by forming carbamate and bicarbonate:

Carbamate formation (primary and secondary amino group)

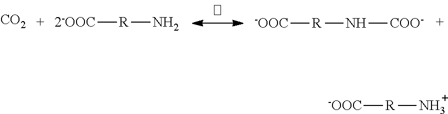

### Carbamate Hydrolysis

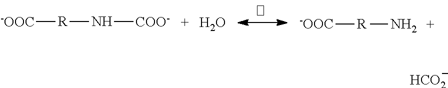

Bicarbonate formation (tertiary amino group, sterically hindered secondary amino group)

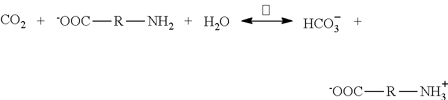

For derivatives of amino acids lacking a carboxyl group, such as taurine:

CO₂⁺K⁻O₃SRNH [symbol] CO₂+⁺K⁻O₃SRNH₂⁺COO⁻

B+⁺K⁻O₃SRNH₂⁺COO⁻ [symbol] BH⁺+⁺K⁻O₃SRNHCOO⁻, wherein B refers to a base.

Another feature of amino acid based solutions is that, as CO₂ reacts with the compound, the product may form precipitates. The presence of solids in the absorption solution can enable a shift of the chemical reaction equilibria resulting in a constant CO₂ pressure when the loading of the solution increases.

To take advantage of the stability, low vapour pressure, biodegradability and favourable energetics for desorption of slow amino acids, such as tertiary amino acids, it would be advantageous to use the solution with an absorption promoter. However, various promoters such as MEA amine would result in higher desorption energy and would thus have drawbacks in the overall CO₂ capture process.

Biocatalysts have also been used for CO₂ absorption. More specifically, CO₂ hydration may be catalyzed by the enzyme carbonic anhydrase as follows:

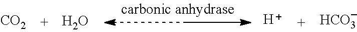

Under optimum conditions, the catalyzed turnover rate of this reaction may reach 1×10⁶ molecules/second.

Carbonic anhydrase has been used as an absorption promoter in amine based solutions to increase the rate of CO₂ absorption. Indeed, particular focus has been on conventional capture processes, that is on amine solutions in conjunction with carbonic anhydrase. In addition to being the most widely studied and applied capture process, an additional reason why amine solutions have been favoured for catalytic enhancement is that they have relatively low ionic strengths, which is a property viewed as significant for carbonic anhydrase hydration activity, since high ionic strength could be detrimental to the stability and function of the protein.

However, amine based solutions can be prone to degradation and oxidation, are not biodegradable, and have high vapour pressures. There is a need for a technology that overcomes at least some of these disadvantages, and offers an improvement in the field of CO₂ capture.

### SUMMARY OF THE INVENTION

The present invention responds to the above mentioned need by providing a formulation and a process for CO₂ capture using amino acids and biocatalysts.

The present invention provides a process for capturing CO₂ from a CO₂-containing gas comprising contacting the CO₂-containing gas with water, biocatalyst and an amino acid compound, enabling the dissolution and transformation of the CO₂ into bicarbonate ions and hydrogen ions, thereby producing an ion-rich solution and a CO₂-depleted gas.

The present invention also provides a formulation for capturing CO₂ from a CO₂-containing gas comprising: water for allowing dissolution of CO₂ therein; biocatalyst for enhancing dissolution and transformation of the CO₂ into bicarbonate and hydrogen ions into the water; and an amino acid compound in the water available for enhancing the transformation of CO₂ catalyzed by the biocatalyst, allowing dissolution of CO₂ and for reacting with CO₂.

The present invention also provides a system for capturing CO₂ from a CO₂-containing gas. The system comprises an absorption unit comprising a gas inlet for the CO₂-containing gas, a liquid inlet for providing an absorption mixture comprising water, biocatalyst and an amino acid compound, the absorption mixture enabling the dissolution and transformation of the CO₂ into bicarbonate ions and hydrogen ions, thereby producing an ion-rich solution and a CO₂-depleted gas. The system comprises a reaction chamber for receiving the absorption mixture and the CO₂-containing gas, in which the dissolution and transformation of CO₂ into bicarbonate and hydrogen ions occurs. The system optionally comprises a gas outlet for expelling the CO₂-depleted gas and a liquid outlet for expelling the ion-rich mixture. The system optionally comprises a regeneration unit for receiving the ion-rich solution and allowing desorption or mineral carbonation to produce an ion-depleted solution. The ion-depleted solution may be recycled back into the absorption unit.

In one optional aspect, the amino acid compound and the biocatalyst may be selected such that the biocatalysts comprise active sites benefiting from removal of protons and the amino acid compounds capture the protons from the biocatalysts to enhance the transformation of the CO₂ into the bicarbonate ions and hydrogen ions.

In another optional aspect, the biocatalysts comprise metalloenzymes, preferably carbonic anhydrase or an analogue thereof.

In another optional aspect, the process comprises performing desorption or mineral carbonation of the ion-rich solution by releasing the bicarbonate ions from the ion-rich solution to produce a CO₂ stream or a mineral and an ion-depleted solution.

In another optional aspect, the amino acid compound comprises at least one primary, secondary and/or tertiary amino acid, derivative thereof, salt thereof and/or mixture thereof.

In another optional aspect, the amino acid compound comprises at least one of the following: glycine, proline, arginine, histidine, lysine, aspartic acid, glutamic acid, methionine, serine, threonine, glutamine, cysteine, asparagine, valine, leucine, isoleucine, alanine, valine, tyrosine, tryptophan, phenylalanine; taurine, N,cyclohexyl 1,3-propanediamine, N-secondary butyl glycine, N-methyl N-secondary butyl glycine, dimethylglycine, sarcosine, methyl taurine, methyl-α-aminopropionic acid, N-(β-ethoxy)taurine, N-(β-aminoethyl)taurine, N-methyl alanine, 6-aminohexanoic acid; or alkali salts thereof; or a combination thereof.

In another optional aspect, the amino acid compound comprises an alkali salt of glycine. In another optional aspect, the amino acid compound comprises an alkali salt of L-methionine. In another optional aspect, the amino acid compound comprises an alkali salt of taurine. In another optional aspect, the amino acid compound comprises an alkali salt of N,N dimethylglycine. In another optional aspect, the amino acid compound comprises an alkali salt of proline.

In another optional aspect, the amino acid compounds are non volatile.

In another optional aspect, the amino acid compound comprises no side chain alcohol groups.

In another optional aspect, the amino acid compound has hydrophilic-hydrophobic properties promoting hydrogen bond stability.

In another optional aspect, the amino acid compound is a sodium or potassium salt of an amino acid, the salt and the amino acid being selected to promote precipitation of precipitates.

In another optional aspect, the amino acid compound is provided in a concentration between about 0.1M and about 6M.

In another optional aspect, the biocatalysts are provided free in the water; dissolved in the water; immobilized on the surface of supports that are mixed in the water and flow therewith; immobilized on the surface of supports that are fixed within an absorption reactor; entrapped or immobilized by or in porous supports that are mixed in the water; entrapped or immobilized by or in porous supports that are fixed within an absorption reactor; as cross-linked enzyme aggregates (CLEA); and/or as cross linked enzyme crystals (CLEC); or a combination thereof.

In another optional aspect, the biocatalysts are supported by micro-particles that are carried with the water.

In another optional aspect, the amino acid compounds have a pKa between about 8 and about 12.5.

In another optional aspect, the amino acid compounds have a pKa above about 9.

In another optional aspect, the amino acid compounds are tertiary amino acids or derivatives thereof. The amino acids may also be others presenting slow absorption kinetics but having elevated stability.

### BRIEF DESCRIPTION OF THE DRAWINGS

FIG. 1 is a process diagram of an embodiment of the present invention, wherein biocatalytic particles or enzymes flow in the absorption solution.

FIG. 2 is a process diagram of another embodiment of the present invention, wherein an absorption unit is coupled to a desorption unit and biocatalytic particles flow in the absorption solution.

FIG. 3 is a graph of relative CO₂ transfer rate for 500 mg/L (human carbonic anhydrase type II) HCAII in K-glycinate solutions at concentrations of 0.1, 0.25 and 0.5 M.

FIG. 4 is a graph of relative CO₂ transfer rate for 500 mg/L HCAII in K-taurate solutions at concentrations of 0.1, 0.25 and 0.5 M.

FIG. 5 is a graph of relative CO₂ transfer rate for 500 mg/L HCAII in solutions of potassium salt of N,N-dimethylglycine at concentrations of 0.1, 0.25 and 0.5 M.

FIG. 6 is a graph showing the impact of the enzyme on CO₂ transfer rate in K-glycinate solutions with an enzyme concentration of 0.5 g/L at a temperature of 20° C.

FIG. 7 is a graph showing residual activity of enzyme micro-particles exposed to MDEA 2M at 40° C., to illustrate stability effects.

### DESCRIPTION OF PREFERRED EMBODIMENTS OF THE INVENTION

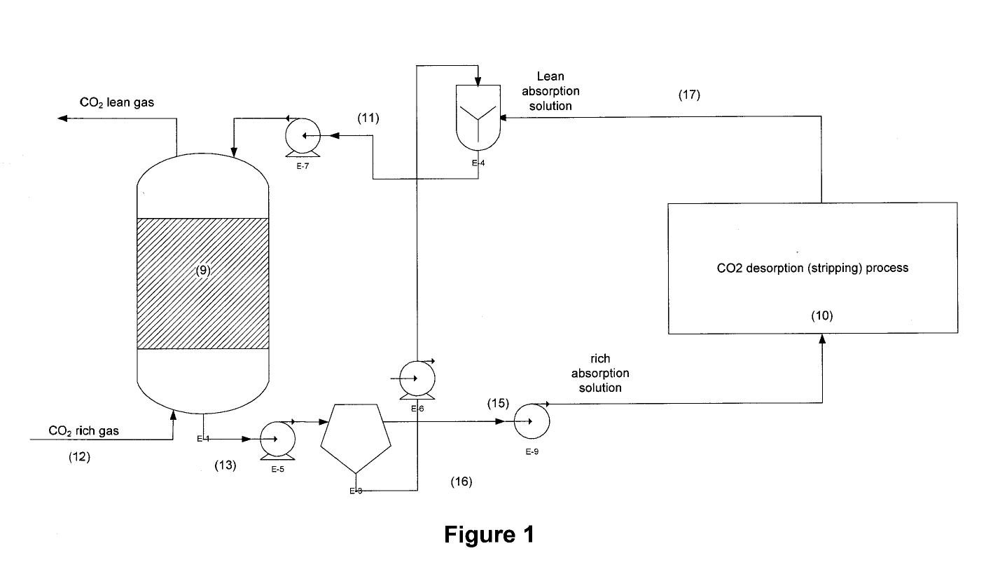

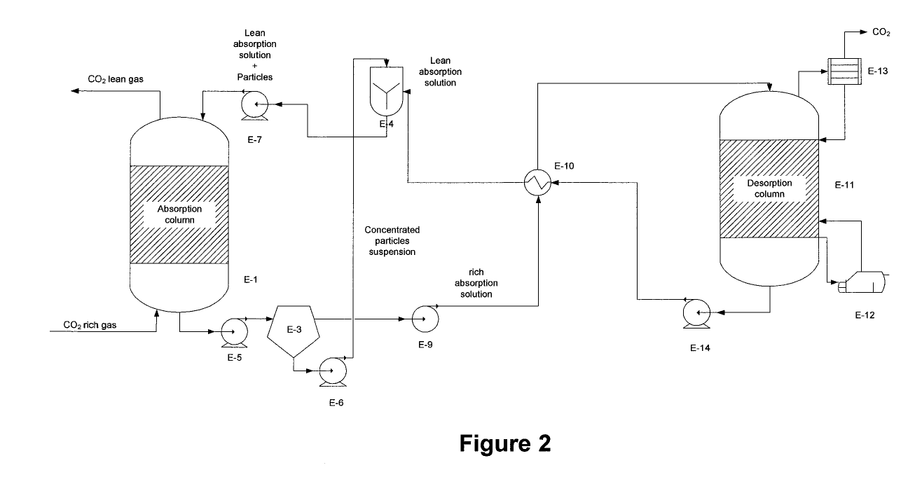

FIGS. 1 and 2 respectively show two different embodiments of the process and system of the present invention. It should also be understood that embodiments of the formulation of the present invention may be used in conjunction with the process and system.

In one aspect of the present invention, the formulation comprises water for allowing dissolution of CO₂, a biocatalyst such as carbonic anhydrase for catalyzing the transformation of CO₂ into bicarbonate and hydrogen ions, and an amino acid compound for reacting with CO₂ to form bicarbonate, and in some cases carbamate ions, allowing CO₂ dissolution and for enhancing the transformation of CO₂ catalyzed by the biocatalyst. The three components may be provided as a pre-mixed solution or mixed on site during the CO₂ capture operation. The absorption step of the CO₂ capture process is improved due to the catalytic ability of the biocatalyst in the presence of the amino acid compound. This improvement aids in enhancing the overall CO₂ capture process as described below.

Considering the case of biocatalysts such as metalloenzymes (e.g carbonic anhydrase), such biocatalysts benefit from a base to promote the capture of H⁺ from each active site to enable it to rapidly react with CO₂ molecules. If the enzyme is used in water only, the CO₂ absorption occurs quite slowly, since the H⁺ is not rapidly transferred and captured.

On the other hand, if only amino acids are used in water, absorption will occur faster than in water only, but generally slower than primary amines such as MEA, resulting in disadvantages such as larger absorber vessels. However, by combining such metalloenzymes and stable but kinetically slower amino acids, enhanced effects are achieved to improve the CO₂ capture process. For instance, the amino acids can both rapidly absorb CO₂ and also capture H⁺ ions from the active sites of the metalloenzymes via at least one amino group of the amino acid, which allows the enzymes to catalyze the hydration reaction of CO₂ in an accelerated manner. This advantageous combination results in smaller absorption equipment and lower energy requirements for desorption than traditional amines, while using a solvent that is more advantageous in terms of stability and biodegradation. For instance, data on DECAB process show that a 6M amino acid salt solution requires 2.3 GJ/ton CO₂ in energy as compared to the MEA process with 4.2 GJ/ton CO₂.

In one preferred aspect of the invention, the amino acids used in the present invention are less volatile than traditional amines. Low volatility of the amino acids results in various improvements such as avoiding evaporative loss which reduces the required makeup in the solution, and reducing the fraction of solution in the gas phase which effectively increases the partial pressure of the CO₂, thereby increasing mass transfer and absorption.

In another preferred aspect of the invention, the amino acids used in the present invention are less susceptible to degradation and are thus more stable than traditional amines. For instance, the amino acids mitigate degradation when the CO₂-containing gas contains other gases such as oxygen that can aggravate degradation of traditional amines.

In another preferred aspect of the invention, the CO₂ capture process is performed within alkaline pH levels such that the amino acids are neutral or negatively charged. In certain alkaline pH conditions, the acid group lacks a proton and the amino group may be neutral or positive. The net charge properties of the amino acids may facilitate certain proton capture mechanisms with the metalloenzyme.

In another preferred aspect of the invention, the amino acids are selected such that they do not contain functional groups that tend to break hydrogen bonds. For instance, the amino acids may contain no side chain alcohol groups that would tend to break hydrogen bonds in the enzyme and denature it. Traditional amines such as MEA have an alcohol group that can disrupt protein structure. Hydrogen bonding occurs between amide groups in secondary protein structure and between “side chains” in tertiary protein structure in a variety of amino acid combinations, both of which can be disrupted by the addition of another alcohol.

In yet another preferred aspect of the invention, the amino acids are selected to have “in between” hydrophilic-hydrophobic properties. The side chains of such amino acids tend to avoid breaking hydrogen bonds in the enzymes. The amino acid compound could also be selected from non-polar amino acids that may be hydrophobic, hydrophilic or in-between, and/or amino acids that have a basic R group.

In various embodiments of the present invention, the amino acid is chosen according to its water-solubility properties, its R group, its acid type and its salts. It should be understood that amino acids of the present invention may include amino sulfonic acids and their salts, e.g. potassium salt of taurate. It should also be understood that “amino acid compound” includes derivatives and variants thereof. It should also be understood that each “amino acid compound” may be a single type of amino acid, a mixture of different amino acids or derivatives or variants thereof, or a compound comprising at least two amino acids which are the same or different, i.e. a polypeptide.

In one embodiment of the invention, the amino acid compound is of the type and is added in sufficient quantities to promote precipitation of an amino species during absorption. The process parameters may be controlled to further promote such precipitation. The amino acid compound may be chosen such that the precipitate has characteristics making it easy to handle with the overall process, by allowing it to be suspended in the reaction solution, pumped, sedimented, etc., as the case may be. The precipitate may be part of the ion-rich solution that is sent for desorption or treated separately for conversion into CO₂ gas. The precipitate may be a bicarbonate species, such as KHCO₃, bicarbonate salts of amino acids (as for proline, sarcosine et β-alanine) or the amino acid itself (as for taurine) and the amino acid salt may be chosen to allow precipitation of such species as desired.

In one embodiment of the invention, the biocatalysts include carbonic anhydrase to enhance performance of absorption solutions for CO₂ capture. The carbonic anhydrase enzyme may be provided directly as part of a formulation or may be provided in a reactor to react with incoming solutions and gases. It should be noted that enzyme used in a free state may be in a pure form or may be in a mixture including impurities or additives such as other proteins, salts and other molecules coming from the enzyme production process. The enzyme may be fixed to a solid non-porous packing material, on or in a porous packing material, on or in particles flowing with the absorption solution within a packed tower or another type of reactor. The carbonic anhydrase may also be in a free state in the formulation or immobilised on particles within the formulation. Immobilized enzyme free flowing in the absorption solution could be entrapped inside or fixed to a porous coating material that is provided around a support that is porous or non-porous. The enzymes may be immobilised directly onto the surface of a support (porous or non porous) or may be present as “cross linked enzymes aggregates” (CLEA) or “cross linked enzymes crystals” (CLEC). CLEA comprises precipitated enzyme molecules forming aggregates that are then crosslinked using chemical agents. The CLEA may or may not have a ‘support’ or ‘core’ made of another material which may or may not be magnetic. CLEC comprises enzyme crystals crosslinked using chemical agents and may also be associated with a ‘support’ or ‘core’ made of another material. When a solid support is used, it may be made of polymer, ceramic, metal(s), silica, solgel, cellulose, chitosan, magnetic particles, and/or other materials known in the art to be suitable for immobilization or enzyme support. When the enzymes are immobilised or provided on particles, such as micro-particles, the particles are preferably sized and provided in a particle concentration such that they are pumpable with the absorption solution. Biocatalysts may also be provided both fixed within the reactor (on a packing material, for example) and flowing with the formulation (as free enzymes, on particles and/or as CLEA or CLEC), and may be the same or different biocatalysts.

The biocatalyst may be provided using means depending on the concentration and type of amino acid compound, the process operating parameters, and other factors. For instance, when a high concentration of amino acid compound is provided, the enzymes may be immobilised on a support to reduce the possibility of deactivation by the amino acid compounds depending on which ones are used. They may be immobilised on porous or non-porous supports, which may be packing mounted within the absorption unit or particles flowing with the solution. In some embodiments, the biocatalyst may be advantageously immobilised in a micro-porous structure allowing access of CO₂ while protecting it against high concentrations of amino acid compounds.

The amino acid compounds used in the formulation may include primary, secondary or tertiary amino acids. The amino acid compounds may more particularly include glycine, proline, arginine, histidine, lysine, aspartic acid, glutamic acid, methionine, serine, threonine, glutamine, cysteine, asparagine, valine, leucine, isoleucine, alanine, valine, tyrosine, tryptophan, phenylalanine, and derivatives such as taurine, N,cyclohexyl 1,3-propanediamine, N-secondary butyl glycine, N-methyl N-secondary butyl glycine, dimethylglycine, sarcosine, methyl taurine, methyl-α-aminopropionic acid, N-(β-ethoxy)taurine, N-(β-aminoethyl)taurine, N-methyl alanine, 6-aminohexanoic acid, etc. and salts thereof.

The amino acids may have a pKa between about 8 and about 12.5. The pKa for tested amines ranged between 7.7 and 9.75.

The amino acid compounds may preferably be stable but “slow” amino acids, such as tertiary amino acids and derivatives thereof. For instance, the amino acids may be diethylglycine or dimethylglycine or another tertiary amino acid.

Carbonic anhydrase enhances performance of amino acid absorption solutions by reacting with dissolved CO₂, thus maintaining a maximum CO₂ concentration gradient between gas and liquid phases and then maximizing CO₂ transfer rate from the gas phase to the absorption solution. The amino acid compounds may also enable the precipitation of bicarbonate species or buffering of hydrogen ions to further improve the CO₂ concentration gradient between gas and liquid phases and thus further increasing CO₂ transfer rate.

The use of amino acids also improves the overall CO₂ capture process by improving desorption of CO₂. The energy consumption required for desorption with amino acids is significantly lower than that generally required for traditional amines such as monoethanolamine (MEA) 5M reference. Thus, the mutual activation of biocatalyst and amino acids improves the absorption and the amino acids further enable lower energy requirements for desorption. In one example, the desorption energy consumption for a 6M amino acid solution was 2.3 GJ/ton CO₂ (DECAB Process) while that for traditional amine MEA 5M reference was 4.2 GJ/ton CO₂, which represents a significant enhancement. In addition, if the enzyme is robust to desorption conditions it could help in accelerating CO₂ desorption and then have an impact on equipment sizing and energy requirements to perform CO₂ desorption.

The following are some advantages, improvements and/or features of some embodiments of the present invention:
The absorption solution is given an increased CO₂ absorption rate.
Introducing carbonic anhydrase into certain amino acid solutions increases absorption rates to levels which will be advantageous over existing amine or amino acid based processes.
The combined increase of CO₂ absorption rates by combining reactivities of amino acids and carbonic anhydrase to enable non volatile, biodegradable but kinetically hindered amino acids coupled with the decrease in the overall energy requirements provides an advantageous overall CO₂ capture process as compared to conventional and enzyme enhanced amine solutions. This is a major step to bringing such technologies to their industrial application in post combustion CO₂ capture.

One embodiment of the process and system is shown in FIG. 1 and will be described in further detail hereafter. To take advantage of biocatalysts flowing in the absorption solution (free or immobilized on/in particles flowing in the absorption solution or as CLEA) for gas scrubbing and especially for CO₂ removal from a CO₂ containing effluent, one process embodiment configuration is shown in FIG. 1. First, the biocatalytic particles are suspended in the lean absorption solution in a mixing chamber (E-4). The biocatalytic particles have a size enabling their flow on, through, and/or around the packing of the packed column without clogging. The lean absorption solution refers to the absorption solution characterized by a low concentration of the species to be absorbed. This solution is either fresh solution or comes from the CO₂ desorption process (1). The absorption solution with biocatalytic particles (11) is then fed to the top of a packed column (E-1) with a pump (E-7). The packing material (9) may be made of conventional material like polymers, metals or ceramics. The geometry of the packing may be chosen from what is commercially available. The packing is preferably chosen to have a geometry or packing arrangement to facilitate the flow of small particles present in the absorption solution. Examples of packing are: Pall rings, Raschig rings, Flexipak, Intalox, Mellapak Plus, etc. Counter-currently, a CO₂ containing gas (12) is fed to the packed column (E-1) and flows on, through, and/or around the packing (9) from the bottom to the top of the column. The absorption solution and biocatalytic particles flow on, through, and/or around the packing material (9) from the top of the column to the bottom. As the absorption solution and biocatalytic particles flow on, through, and/or around the packing, the absorption solution becomes richer in the compound that is being absorbed, in this case CO₂. Biocatalytic particles, present near the gas-liquid interface, enhance CO₂ absorption by immediately reacting with CO₂ to produce bicarbonate ions and protons and thus maximizing the CO₂ concentration gradient across the gas-liquid interface. At the exit of the column, the rich absorption solution and biocatalytic particles (13) are pumped (E-5) to a particle separation unit (E-3). Rich absorption solution refers to the absorption solution characterized by a concentration of absorbed compound which is higher than that of the lean solution. The separation unit may consist of a filtration unit, a centrifuge, a cyclone, a magnetic separator, a sedimentation tank and any other units or equipments known for particles or solids separation. The absorption solution without particles (15) is then pumped (E-9) to another unit which may be a CO₂ desorption unit (10). Biocatalytic particles (16) are pumped (E-6) to a mixing chamber (E-4) where they are mixed with the CO₂ lean absorption solution. The mixing chamber may be equipped with an impeller or another device whose function is to assure that biocatalytic particles are in suspension in the absorption solution which is then pumped (E-7) once again to the absorption column (E-1). In one embodiment, the absorption may be operated between 40-70° C. and desorption between 80-150° C.

In one embodiment, the absorption unit is coupled to a desorption unit as shown in further detail in FIG. 2. In this embodiment, the absorption solution rich in CO₂ without biocatalytic particles (15) is pumped (E-9) through a heat exchanger (E-10) where it is heated and then to the desorption column (E-11). In the desorption unit, the solution is further heated in order that the CO₂ is released from the solution in a gaseous state. Because of relatively high temperature used during desorption, water also vaporizes. Part of the absorption solution (18) is directed toward a reboiler (E-12) where it is heated to a temperature enabling CO₂ desorption. Gaseous CO₂ together with water vapour are cooled down, water condenses and is fed back to the desorption unit (19). Dry gaseous CO₂ (20) is then directed toward a compression and transportation process for further processing. The liquid phase, containing less CO₂, and referred to as the lean absorption solution (17) is then pumped (E-14) to the heat exchanger (E-10) to be cooled down and fed to the mixing chamber (E-4). The temperature of the lean absorption solution (17) should be low enough not to denature the enzyme.

In one embodiment, in addition to amino acid compounds there may also be carbonates and/or amines used in the absorption solution. The carbonate compounds may be potassium carbonate, sodium carbonate, ammonium carbonate, promoted potassium carbonate solutions and promoted sodium carbonate solutions, and such compounds may enable a decrease in the desorption energy required and/or precipitation of other species to accelerate the absorption, among other advantages. In this embodiment of combining amino acids, carbonates and biocatalysts, one may further increase the performance of the formulation, process and system of the present invention. In one preferred embodiment, the amino acid promoter is used in conjunction with the biocatalyst immobilised on a packing in a packed-tower absorption reactor.

In the case that enzymes are free flowing in the absorption solution and are robust to desorption operating conditions, the process may be slightly different from the one shown in FIG. 1. For such a case, units E-3, E-6 and E-9 may not be present since they are required for the processing of the biocatalytic particles in the absorption solution. Unit E-4 would be used to introduce new enzyme in the process.

An aqueous absorption solution of potassium salt of taurate (2-aminoethanesulfonic acid potassium salt) may be used in combination with carbonic anhydrase to enhance its CO₂ absorption performance. The enzyme may be used in any manner as described hereinabove, free or immobilized. For instance, immobilized enzymes may consist of enzyme molecules attached to the surface of a support (porous or non porous), or enzyme molecules entrapped inside the matrix of porous particles, or cross-linked enzymes aggregates (CLEA). The support may consist of tower packing or small particles like beads. In the case of particles, size may be selected in order that they can be suspended and pumped in the potassium salt of taurate solution. The role of the enzyme is to rapidly react with dissolved CO₂ and thus to maximize the CO₂ concentration gradient across the absorption solution and the gas phase containing CO₂. The increase in performance using this amino acid compound with enzymes may depend on the way the enzyme is used. Since the role of the enzyme is to maximize the CO₂ concentration gradient across the gas-liquid interface, the closer the enzyme is to the interface, and the more homogeneously the enzyme is distributed in the solution, the better the impact of the enzyme as an accelerator. Absorption performance of the potassium salt of taurate will be greatest with free enzyme, which would be superior to the performance of enzymes on/in particles, which is equivalent to CLEAs or CLECs, which in turn is superior to enzymes on tower packing, which is greater than no enzyme. The amino acid absorption formulation may include glycine, proline, arginine, histidine, lysine, aspartic acid, glutamic acid, methionine, serine, threonine, glutamine, cysteine, asparagine, valine, leucine, isoleucine, alanine, valine, tyrosine, tryptophan, phenylalanine, and derivatives such as taurine, N,cyclohexyl 1,3-propanediamine, N-secondary butyl glycine, N-methyl N-secondary butyl glycine, diethylglycine, dimethylglycine, sarcosine, methyl taurine, methyl-α-aminopropionic acid, N-(β-ethoxy)taurine, N-(β-aminoethyl)taurine, N-methyl alanine, 6-aminohexanoic acid, etc. and salts thereof.

In one embodiment, the absorption solution containing enzymes (free or particles) forms a solid precipitate as the result of CO₂ absorption and reaction with this absorption solution and with the enzymes. Solid precipitates are removed from the rich absorption solution and then fed to the desorption unit. Removal methods comprise filtration, sedimentation, centrifugation, etc. The lean absorption solution (without solid precipitates) is recycled back to the absorption unit. In this process, free enzyme would not be exposed to desorption (or only a very small fraction). In the case the enzyme is present on/in particles, if enzyme is robust to desorption conditions, particles might be fed with solid precipitates to the desorption. In the event the enzyme is not robust to desorption conditions, particles would have to be separated from solid precipitate and kept in the lean solution.

### EXAMPLES

The following examples present different ways to activate absorption solutions with carbonic anhydrase and generally elaborate on the embodiments of the present invention.

### Example 1

An experiment was conducted in an absorption packed column. The absorption solution is an aqueous solution of potassium taurate (1.5M). This absorption solution is contacted counter-currently with a gas phase with a CO₂ concentration of 130,000 ppm. Liquid flow rate was 0.65 g/min and gas flow rate was 65 g/min corresponding to L/G of 10 (g/g). Gas and absorption solution were at room temperature. Operating pressure of the absorber was set at 1.4 psig. The column has a 7.5 cm diameter and a 50 cm height. Packing material is polymeric Raschig rings 0.25 inch. Two tests were performed: the first with no biocatalyst, the second with carbonic anhydrase immobilized to packing support.

The results obtained showed that CO₂ transfer rate or CO₂ removal rate increased from 83 to 117 mmol CO₂/min with carbonic anhydrase immobilized onto the surface of Raschig rings. These results clearly demonstrate the positive impact of adding the enzyme in a packed column.

### Example 2

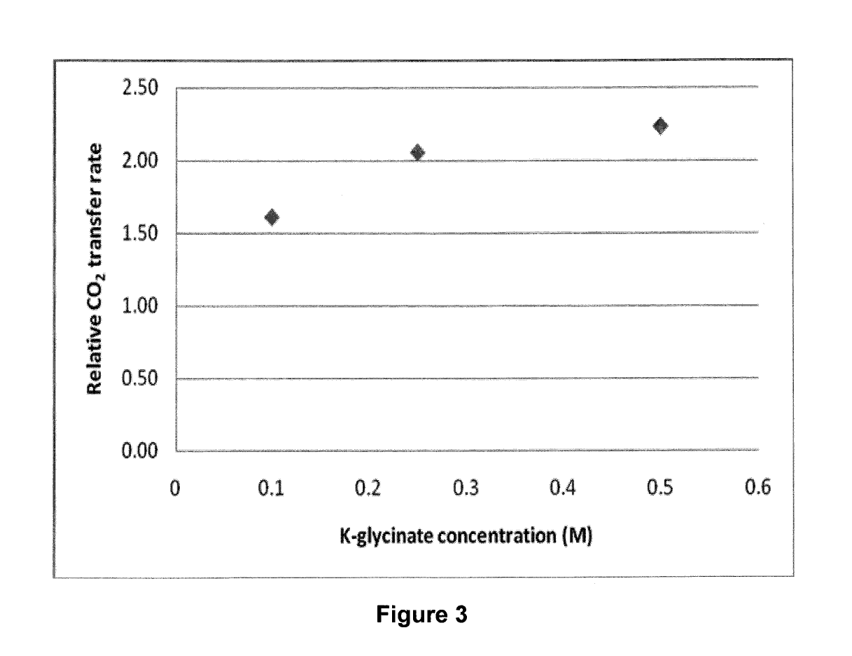

Tests were conducted in a stirred cell at enzyme concentration of 500 mg/L in a potassium glycinate (or potassium salt of glycine) solution at concentrations of 0.1, 0.25 and 0.5 M and at a temperature of 20° C. Enzyme used is human carbonic anhydrase II (HCAII). Initial CO₂ loading is 0 mol/mol. The stirred cell contains the absorption solution (and the enzyme when required). A continuous flow of pure CO₂ is flushed in the stirred cell over the liquid phase and pH change of the solution is monitored. Changes in pH are correlated to changes in inorganic carbon concentration which is used to calculate CO₂ transfer rates. Tests were conducted with and without enzyme to enable determination of the enzyme impact. Results are expressed as a ratio of CO₂ transfer rate with enzyme to CO₂ transfer rate in the absence of the enzyme (see FIG. 3). Results clearly indicate that enzyme brings an important benefit for all tested to the K₂CO₃ solutions.

An additional test was performed with 500 mg/L HCAII in 0.5 M K-glycinate solution at a temperature of 40° C. Results indicate that the impact of the enzyme remains the same as what was observed at 20° C.

### Example 3

Tests were conducted in a stirred cell at enzyme concentration of 500 mg/L in a potassium methionate solution (potassium salt of L-methionine) at concentrations of 0.1, and 0.25 M at a temperature of 20° C. Enzyme used is human carbonic anhydrase II (HCAII). Initial CO₂ loading is 0 mol/mol. The stirred cell contains the absorption solution (and the enzyme when required). A continuous flow of pure CO₂ is flow flushed in the stirred cell over the liquid phase and pH change of the solution is monitored. Changes in pH are correlated to changes in inorganic carbon concentration which is used to calculate CO₂ transfer rates. Tests were conducted with and without enzyme to enable determination of the enzyme impact. Results are expressed as a ratio of CO₂ transfer rate with enzyme to CO₂ transfer rate in the absence of the enzyme (see Table 1). Results clearly indicate that enzyme brings an important benefit for all tests with K-methionate solutions.

TABLE 1
Relative CO₂ transfer rates observed in K-methionate solutions
at 25° C. with an enzyme concentration of 500 mg/L.

| K-methionate concentration | |
|---|---|
| (M) | CO₂ relative transfer rate |
| 0.1 | 1.3 |
| 0.25 | 1.8 |

### Example 4

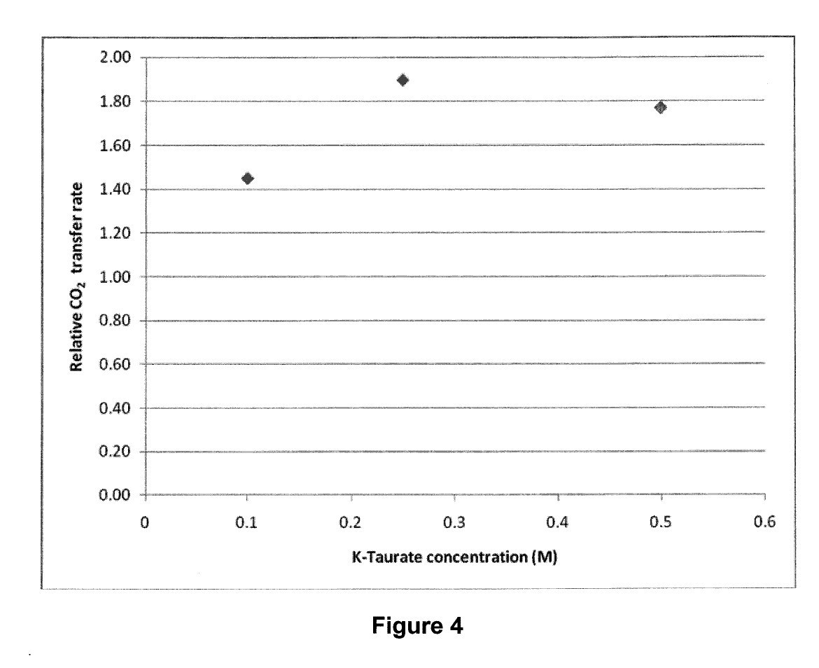

Tests were conducted in a stirred cell at enzyme concentration of 500 mg/L in a potassium taurate solution (potassium salt of taurine) at concentrations of 0.1, 0.25 and 0.5 M at a temperature of 20° C. Enzyme used is human carbonic anhydrase II (HCAII). Initial CO₂ loading is 0 mol/mol. The stirred cell contains the absorption solution (and the enzyme when required). A continuous flow of pure CO₂ is flow flushed in the stirred cell over the liquid phase and pH change of the solution is monitored. Changes in pH are correlated to changes in inorganic carbon concentration which is used to calculate CO₂ transfer rates. Tests were conducted with and without enzyme to enable determination of the enzyme impact. Results are expressed as a ratio of CO₂ transfer rate with enzyme to CO₂ transfer rate in the absence of the enzyme (see FIG. 4). Results clearly indicate that enzyme brings an important benefit for all tests with K-taurate solutions.

### Example 5

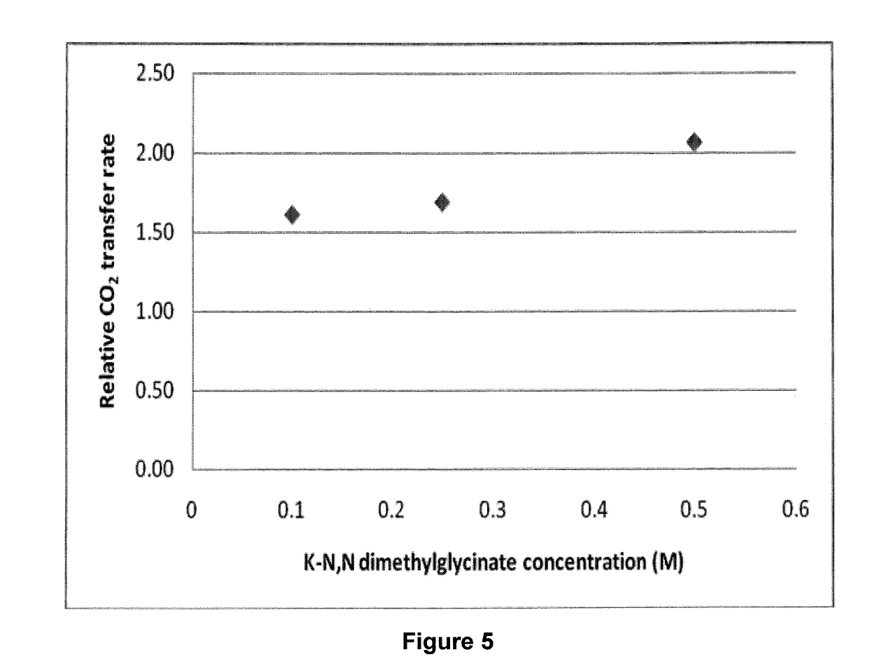

Tests were conducted in a stirred cell at an enzyme concentration of 500 mg/L in a solution of potassium salt of N,N-dimethylglycine at concentrations of 0.1, 0.25 and 0.5 M at a temperature of 20° C. Enzyme used is human carbonic anhydrase II (HCAII). Initial CO₂ loading is 0 mol/mol. Method is as described in Example 2. Results, shown in FIG. 5, clearly indicate that enzyme brings an important benefit for all tested concentrations.

### Example 6

To determine the impact of enzyme particles on CO₂ absorption rate tests were also conducted in a stirred cell. This device is used to evaluate impact of enzyme particles on the CO₂ absorption rate in a given absorption solution. Tests are conducted as follows: a known volume of the unloaded absorption solution is introduced in the reactor, then a known mass of particles are added to the absorption solution (particles may or may not contain enzyme), a CO₂ stream is flowed through the head space of the reactor and agitation is started. pH of the solution is measured as a function of time. Then pH values are converted into carbon concentration in g C/L using a carbon concentration-pH correlation previously determined for the absorption solution. Absorption rates are determined from a plot of C concentration as a function of time and the impact of the enzyme is reported as the ratio of absorption rate in the presence of the enzyme particles to absorption rate in the presence of particles without enzyme.

### Example 7

Tests were conducted with HCAII immobilised (non-optimised protocol) at the surface of nylon micro-particles. Nylon particle size ranges between 50 and 160 microns. Absorption solution were 0.5 M of the potassium salt of the following amino acids: glycine, methionine, taurine and N,N-dimethylglycine. Testing temperature was 20° C. Enzyme concentration is 0.5 g/L. Method is described in Example 6. Results indicate that enzyme on nylon micro-particles increases CO₂ absorption rate for all tested amino acid salts (see Table 2).

TABLE 2
Relative CO₂ transfer rates in presence of enzyme immobilized
on nylon particles in 0.5M potassium salt of amino
acids at enzyme concentration of 0.5 g/L

| Amino acid | Relative transfer rate |
|---|---|
| Glycine | 1.4 |
| Methionine | 1.5 |
| Taurine | 1.6 |
| N,N-dimethylglycine | 1.1 |

### Example 8

A comparison was made on the impact of the enzyme measured in a 0.5 M potassium salt of taurine (K-Taurine) solution to that obtained in a sodium salt of taurine (Na-Taurine). Both tests were conducted with 0.5 g/L carbonic anhydrase at a temperature of 20° C. Tests were run in a stirred cell (see Example 2). Results are shown in Table 3. It can be observed that enzyme has a similar relative impact in both solutions.

TABLE 3
Impact of the presence of 0.5 g/L carbonic anhydrase in
0.5M K-Taurate and 0.5M Na-Taurate solutions at 20° C.

| | Relative transfer |
|---|---|
| Solution | rate |
| K-Taurate | 1.8 |
| Na-Taurate | 1.7 |

### Example 9

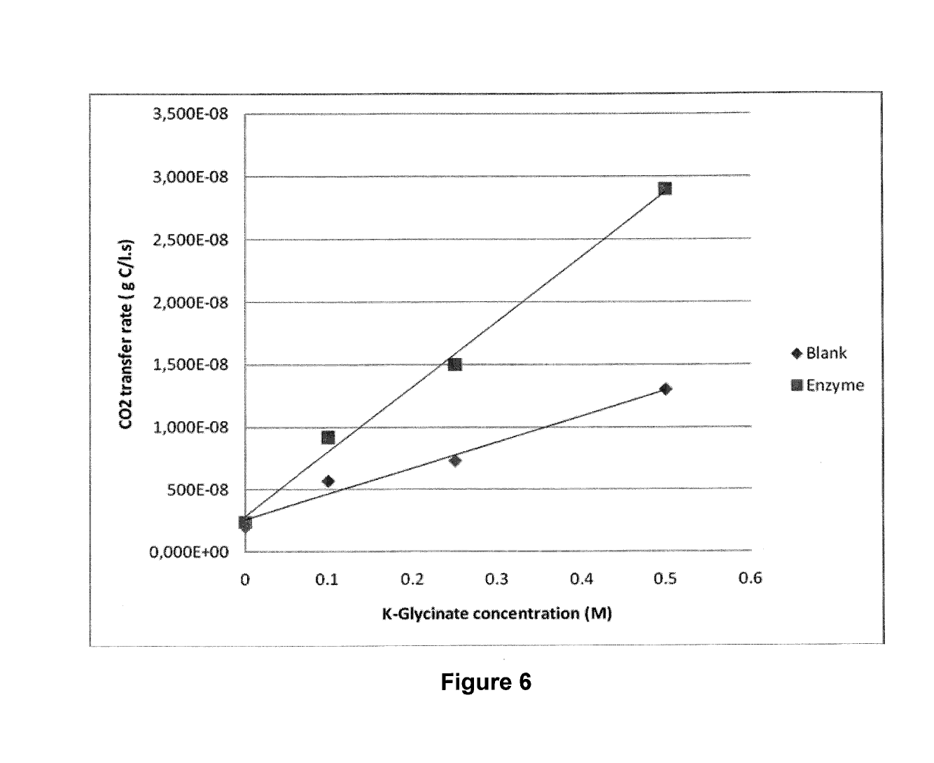

The impact of carbonic anhydrase was compared for different solutions of the potassium salt of glycine at concentrations of 0, 0.1, 0.25 and 0.5 M. To consider that amino acid solutions are alkaline, the zero concentration was prepared with water by adjusting pH to 12 using NaOH, which was the highest pH observed of the various amino acids tested. Then for each solution, CO₂ transfer rate was measured in a stirred cell (Example 6) in absence of carbonic anhydrase and in presence of an enzyme concentration of 0.5 g/L. Tests were conducted at 20° C. Results are shown in FIG. 6. It can be observed that for a similar pH, presence of the potassium salt of amino acid increases CO₂ transfer rate. It can also be observed that the CO₂ transfer rate increases as solution concentration is higher. Addition of the enzyme to these solutions resulted in all cases in an increase in CO₂ transfer rates. Increases in CO₂ transfer because of the presence of the enzyme are higher at higher solution concentrations and seem to be proportional to the solution concentration under the tested conditions.

### Example 10

The impact of CO₂ loading of a given solution on the impact of the enzyme was tested for 0.5 M K-glycinate, 0.25 K-L-methionate, 0.5 M K-Taurate and K-N,N-dimethylglycinate solutions at a temperature of 20° C. Transfer rates with and without enzyme were evaluated considering results previously obtained for those solutions (see Examples 2-4 and 5). Loading values at which the impact of the enzyme was determined are found in Table 4 with other results.

TABLE 4
Impact of carbonic anhydrase for different amino
acid solutions at two CO₂ loading values

| | CO₂ loading | |
|---|---|---|
| Solution | (mol CO₂/mol amino acid) | Relative transfer rate |
| 0.5M K-glycinate | 0 | 2.2 |
| | 0.4 | 2.4 |
| 0.25M K-L-methionate | 0 | 1.8 |
| | 0.5 | 1.3 |
| 0.5M K-taurate | 0 | 1.8 |
| | 0.4 | 1.6 |
| 0.5M K-N,N dimethylglycinate | 0 | 2.1 |
| | 0.2 | 2.2 |

Results indicate that enzyme continues to have a significant impact at higher CO₂ loadings.

### Example 11

This example provides data to demonstrate that enzyme immobilization increases enzyme stability. Data are shown for enzyme immobilized on nylon micro-particles

To evaluate the impact of immobilization on enzyme stability, the stability of immobilized enzymes was evaluated and compared to the stability of the same enzyme in a soluble form.

Non-limiting example of nylon micro-particles:

Micro-particles were prepared through the following non-optimized steps:
Surface treatment of nylon micro-particles with glutaraldehyde
Addition of polyethyleneimine
Addition of glutaraldehyde
Enzyme fixation (human carbonic anhydrase type II)
Aldehyde group blocking with polyethyleneimine

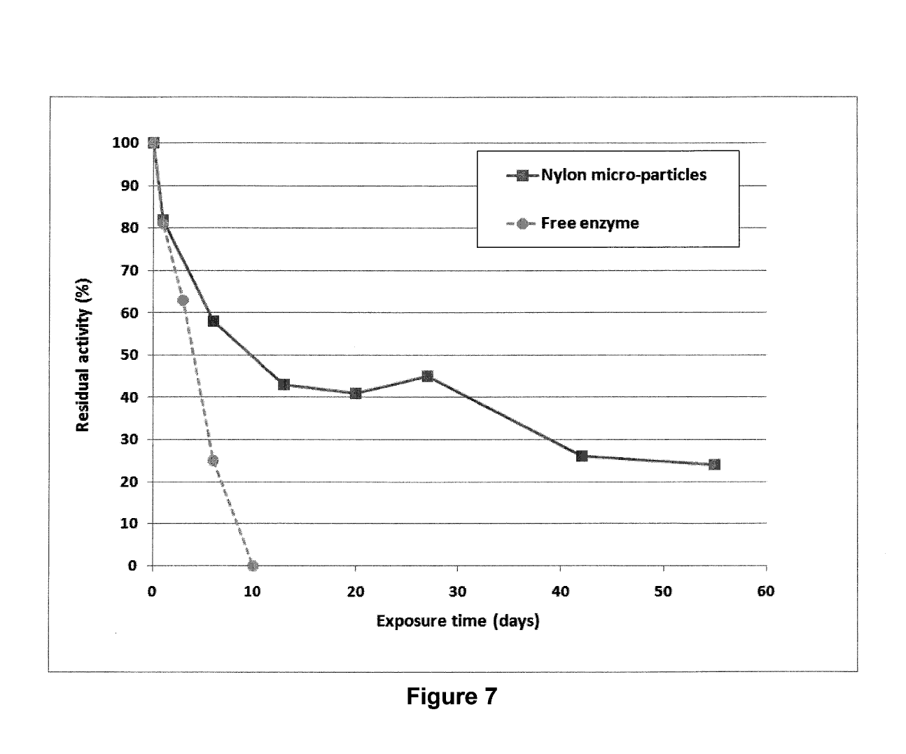

Following immobilization, the enzyme micro-particles and soluble enzyme were exposed to MDEA 2M at 40° C. At specific exposure times, samples were withdrawn and activity was measured. Results are expressed as residual activity, which is the ratio of the activity of the enzyme at a given exposure time t to the enzyme activity at time 0. FIG. 7 illustrates the results.

Results show that free enzyme loses all activity with 10 days, whereas micro-particles still retain 40% residual activity after 56 days. From this result, it is clear that immobilization increases enzyme stability under these conditions.

These results show the potential of immobilization to increase the stability of carbonic anhydrase at higher temperature conditions that are found in a CO₂ capture process. These results were obtained in MDEA 2M at 40° C. and it is expected that a similar increase in stability will also be present in amino acid solutions. In optional aspects of the present invention, the biocatalyst is provided to enable increased stability around or above the stability increase illustrated in the examples.

It should also be noted that the absorption and desorption units that may be used with embodiments of the present invention can be different types depending on various parameters and operating conditions. The reactor types may be chosen depending on the presence of free biocatalysts, biocatalytic micro-particles, biocatalytic fixed packing, etc. The units may be, for example, in the form of a packed reactor, spray reactor, fluidised bed reactor, etc., may have various configurations such as vertical, horizontal, etc., and the overall system may use multiple units in parallel or in series, as the case may be.

It should be understood that the embodiments described and illustrate above do not restrict what has actually been invented.

1. A process for capturing CO₂ from a CO₂-containing gas, comprising:
contacting the CO₂-containing gas with water, biocatalysts and an amino acid compound, enabling the dissolution and transformation of the CO₂ into bicarbonate ions and hydrogen ions, thereby producing an ion-rich solution and a CO₂-depleted gas.

2. The process of claim 1, wherein the amino acid compound and the biocatalysts are selected such that the biocatalysts comprise active sites benefiting from removal of protons and the amino acid compounds capture the protons from the biocatalysts to enhance the transformation of the CO₂ into the bicarbonate ions and hydrogen ions.

3. The process of claim 1, wherein the biocatalysts comprise metalloenzymes.

4. The process of claim 1, wherein the biocatalysts comprise carbonic anhydrase or an analogue thereof.

5. The process of claim 1, comprising
performing desorption or mineral carbonation of the ion-rich solution by releasing the bicarbonate ions from the ion-rich solution to produce a CO₂ stream or a mineral and an ion-depleted solution.

6. The process of claim 1, wherein the amino acid compound comprises at least one primary, secondary and/or tertiary amino acid, derivative thereof, salt thereof and/or mixture thereof.

7. The process of claim 6, wherein the amino acid compound comprises at least one of the following: glycine, proline, arginine, histidine, lysine, aspartic acid, glutamic acid, methionine, serine, threonine, glutamine, cysteine, asparagine, valine, leucine, isoleucine, alanine, valine, tyrosine, tryptophan, phenylalanine; taurine, N,cyclohexyl 1,3-propanediamine, N-secondary butyl glycine, N-methyl N-secondary butyl glycine, diethylglycine, dimethylglycine, sarcosine, methyl taurine, methyl-α-aminopropionic acid, N-(β-ethoxy)taurine, N-(β-aminoethyl)taurine, N-methyl alanine, 6-aminohexanoic acid; or alkali salts thereof; or a combination thereof.

8. The process of claim 1, wherein the amino acid compound comprises an alkali salt of glycine.

9. The process of claim 1, wherein the amino acid compound comprises an alkali salt of L-methionine.

10. The process of claim 1, wherein the amino acid compound comprises an alkali salt of taurine.

11. The process of claim 1, wherein the amino acid compound comprises an alkali salt of N,N dimethylglycine.

12. The process of claim 1, wherein the amino acid compound comprises an alkali salt of proline.

13. The process of claim 1, wherein the amino acid compounds are non volatile.

14. The process of claim 1, wherein the amino acid compound comprises no side chain alcohol groups.

15. The process of claim 1, wherein the amino acid compound has hydrophilic-hydrophobic properties promoting hydrogen bond stability.

16. The process of claim 1, wherein the amino acid compound is a salt of an amino acid, the salt and the amino acid being selected to promote precipitation of precipitates.

17. The process of claim 1, wherein the amino acid compound is provided in a concentration between about 0.1M and about 6M.

18. The process of claim 1, wherein the biocatalysts are provided free in the water; dissolved in the water; immobilized on the surface of supports that are mixed in the water and flow therewith; immobilized on the surface of supports that are fixed within an absorption reactor; entrapped or immobilized by or in porous supports that are mixed in the water; entrapped or immobilized by or in porous supports that are fixed within an absorption reactor; as cross-linked enzyme aggregates (CLEA); and/or as cross linked enzyme crystals (CLEC); or a combination thereof.

19. The process of claim 1, wherein the biocatalysts are supported by micro-particles that are carried with the water.

20. The process of claim 1, wherein the amino acid compounds have a pKa between about 8 and about 12.5.

21. The process of claim 1, wherein the amino acid compounds have a pKa above about 9.

22. The process of claim 1, wherein the amino acid compounds are tertiary amino acids or derivatives thereof.

23. A formulation for capturing CO₂ from a CO₂-containing gas comprising:
water for allowing dissolution of CO₂ therein;
biocatalysts for enhancing dissolution and transformation of the CO₂ into bicarbonate and hydrogen ions into the water;
an amino acid compound in the water available for enhancing the transformation of CO₂ catalyzed by the biocatalysts.

24. The formulation of claim 23, wherein the amino acid compound and the biocatalysts are selected such that the biocatalysts comprise active sites benefiting from removal of protons and the amino acid compounds capture the protons from the biocatalysts to enhance the transformation of the CO₂ into the bicarbonate ions and hydrogen ions.

25. The formulation of claim 23, wherein the biocatalysts comprise metalloenzymes.

26. The formulation of claim 23, wherein the biocatalysts comprise carbonic anhydrase or an analogue thereof.

27. The formulation of claim 23, wherein the amino acid compound comprises at least one primary, secondary and/or tertiary amino acid, derivative thereof, salt thereof and/or mixture thereof.

28. The formulation of claim 27, wherein the amino acid compound comprises at least one of the following: glycine, proline, arginine, histidine, lysine, aspartic acid, glutamic acid, methionine, serine, threonine, glutamine, cysteine, asparagine, valine, leucine, isoleucine, alanine, valine, tyrosine, tryptophan, phenylalanine, taurine, N,cyclohexyl 1,3-propanediamine, N-secondary butyl glycine, N-methyl N-secondary butyl glycine, diethylglycine, dimethylglycine, sarcosine, methyl taurine, methyl-α-aminopropionic acid, N-(β-ethoxy)taurine, N-(β-aminoethyl)taurine, N-methyl alanine, 6-aminohexanoic acid; alkali salt thereof; or a combination thereof.

29. The formulation of claim 23, wherein the amino acid compound comprises an alkali salt of glycine.

30. The formulation of claim 23, wherein the amino acid compound comprises an alkali salt of L-methionine.

31. The formulation of claim 23, wherein the amino acid compound comprises an alkali salt of taurine.

32. The formulation of claim 23, wherein the amino acid compound comprises an alkali salt of N,N dimethylglycine.

33. The formulation of claim 23, wherein the amino acid compound comprises an alkali salt of proline.

34. The formulation of claim 23, wherein the amino acid compounds are non volatile.

35. The formulation of claim 23, wherein the amino acid compound comprises no side chain alcohol groups.

36. The formulation of claim 23, wherein the amino acid compound has hydrophilic-hydrophobic properties promoting hydrogen bond stability.

37. The formulation of claim 23, wherein the amino acid compound is a sodium or potassium salt of an amino acid, the salt and the amino acid being selected to promote precipitation of precipitates.

38. The formulation of claim 23, wherein the amino acid compound is provided in a concentration between about 0.1 and about 6M.

39. The formulation of claim 23, wherein the biocatalyst activator is provided free in the water; dissolved in the water; immobilized on the surface of supports that are mixed in the water and are flowable therewith; entrapped or immobilized by or in porous supports that are mixed in the water and are flowable therewith; as cross-linked aggregates or crystals; or a combination thereof.

40. The formulation of claim 23, wherein the amino acid compounds have a pKa between about 8 and about 12.5.

41. The formulation of claim 23, wherein the amino acid compounds have a pKa above about 9.

42. The formulation of claim 23, wherein the amino acid compounds are tertiary amino acids or derivatives thereof.
